(19) United States
(12) Patent Application Publication
(10) Pub. No.: US 2007/0010889 A1
(43) Pub. Date: Jan. 11, 2007

(54) Foldable nucleus replacement device

(75) Inventor: Tom J. Francis, Cordova, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI HOLDINGS, INC.

(21) Appl. No.: 11/174,617

(22) Filed: Jul. 6, 2005

(51) Int. Cl. A61F 2/44

(52) U.S. Cl. 623/17.16; 623/17.11

(57) ABSTRACT

Embodiments include a foldable nucleus replacement device comprising a body capable of folding upon itself more than twice at pre-determined folds. Each fold in the device may define an inner surface and an outer surface. The body of the device also may be capable of unfolding to an approximately linear state before implantation, and returning to its folded, non-linear state after implantation. In this way, the foldable nucleus replacement device may be unfolded to its approximately linear state having a smaller cross section for implantation, and then return to its folded, non-linear state to function as a nucleus replacement after implantation. Additionally, embodiments include a method for replacing at least a portion of an intervertebral disc nucleus using foldable nucleus replacement devices such as those disclosed herein.

### FIELD OF THE INVENTION

Embodiments of the invention relate to prostheses for replacement and augmentation of the nucleus in an intervertebral disc. More particularly, embodiments of the invention relate to foldable nucleus replacement devices.

### BACKGROUND

The intervertebral disc functions to stabilize the spine and to distribute forces between vertebral bodies. The intervertebral disc is composed primarily of three structures: the nucleus pulposus, the annulus fibrosis, and two vertebral end-plates. These components work together to absorb the shock, stress, and motion imparted to the spinal column. The nucleus pulposus is an amorphous hydrogel in the center of the intervertebral disc. The annulus fibrosis, which is composed of highly structured collagen fibers, surrounds and maintains the nucleus pulposus within the center of the intervertebral disc. The vertebral end-plates, composed of hyalin cartilage, separate the disc from adjacent vertebral bodies and act as a transition zone between the hard vertebral bodies and the soft disc.

Intervertebral discs may be displaced or damaged due to trauma or disease. Disruption of the annulus fibrosis may allow the nucleus pulposus to protrude into the vertebral canal, a condition commonly referred to as a herniated or ruptured disc. The extruded nucleus pulposus may press on a spinal nerve, resulting in nerve damage, pain, numbness, muscle weakness, and paralysis. Intervertebral discs also may deteriorate due to the normal aging process. As a disc dehydrates and hardens, the disc space height may be reduced, leading to instability of the spine, decreased mobility, and pain.

One way to relieve the symptoms of these conditions is by surgical removal of a portion or all of the intervertebral disc. The removal of the damaged or unhealthy disc may allow the disc space to collapse, however, which might lead to instability of the spine, abnormal joint mechanics, nerve damage, and severe pain. Therefore, it has been proposed that a prosthesis be implanted in order to replace all or a portion of the damaged disc.

One such prosthesis is a nucleus replacement device for replacement or augmentation of the nucleus pulposus. Nucleus replacements may be used when the nucleus pulposus of the intervertebral disc is damaged but the annulus fibrosis and vertebral end-plates are still sufficiently healthy to retain. Nucleus replacement surgery typically involves removing the damaged nucleus pulposus of the intervertebral disc and insertion of the nucleus replacement device inside of the retained annulus fibrosis. Some desirable attributes of a hypothetical nucleus replacement device include axial compressibility for shock absorbance, excellent durability to avoid future replacement, and bio-compatibility.

An example of a nucleus replacement device is disclosed in U.S. Pat. No. 6,620,196, incorporated herein by reference in its entirety, which discloses an implant configurable in two positions: (i) a straightened state for insertion through a small opening in the annulus; and (ii) a folded state wherein the implant folds into a kidney shape similar to that of a natural nucleus pulposus. The foldable implant is molded from a polymer and may have several distinct structures, such as a foldable core, outer shell, and reinforcement means.

The description herein of problems and disadvantages of known apparatus, methods, and devices is not intended to limit the invention to the exclusion of these known entities. Indeed, embodiments of the invention may include one or more of the known apparatus, methods, and devices without suffering from the disadvantages and problems noted herein.

### SUMMARY OF THE INVENTION

There is a need for an improved nucleus replacement device. More particularly, there is a need for a nucleus replacement device that is capable of conforming to a configuration having a substantially smaller cross section for implantation, and that transforms to a different configuration upon implantation having a substantially larger cross section. There also is a need for a nucleus replacement device that can be subjected to significant short-term deformation without significant damage to or change in its long-term performance. Embodiments of the invention solve some or all of these needs, as well as additional needs.

Therefore, in accordance with an embodiment of the present invention, there is provided a foldable nucleus replacement device comprising a body capable of folding upon itself more than twice at pre-determined folds, each fold defining an inner surface and an outer surface. The body also may be capable of unfolding to an approximately linear state before implantation, and returning to its folded, non-linear state after implantation.

In accordance with another embodiment of the present invention, there is provided a method for replacing at least a portion of an intervertebral disc nucleus. A foldable nucleus replacement device as described herein may be provided. The foldable nucleus replacement device may be unfolded to an approximately linear state and inserted into an intervertebral disc space. During insertion, the nucleus replacement device may be allowed to return to its folded, non-linear state.

These and other features and advantages of the present invention will be apparent from the description provide herein.

### BRIEF DESCRIPTION OF THE DRAWINGS

FIG. 1, embodiments A (plan view) and B (perspective view), is a drawing of an exemplary foldable nucleus replacement device.

FIG. 2 is a side view of an exemplary foldable nucleus replacement device.

### DETAILED DESCRIPTION OF THE EMBODIMENTS

The following description is intended to convey a thorough understanding of the various embodiments of the invention by providing a number of specific embodiments and details involving foldable nucleus replacement devices. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

Throughout this description, the term “folding” and other derivatives of the term “fold” refer to the ability of an object to flex to an approximately 180 degree angle, preferably from 90 degrees to 180 degrees or more, and most preferably from 170 degrees to 190 degrees. Folding does not encompass a spring or coil-type device that uncoils or coils during or after insertion.

The expression “pre-determined folds” means that the foldable nucleus replacement device's body is capable of folding preferentially at pre-determined points or locations in the body. For example, the pre-determined folds may have indents, grooves, or cutouts on the inner and outer surfaces defined by the fold, the indents, grooves, or cutouts enabling the folding of the body at that particular point or location.

It is a feature of an embodiment of the present invention to provide a foldable nucleus replacement device comprising a body capable of folding upon itself more than twice at pre-determined folds, each fold defining an inner surface and an outer surface. The body preferably may be capable of unfolding to an approximately linear state before implantation, and returning to its folded, non-linear state after implantation.

Preferably, the device's body is sufficiently deformable so as to be capable of being unfolded to an approximately linear state for at least a temporary period of time without significant permanent deformation, cracks, tears, breakage, or other damage. The approximately linear state of the device's body may be advantageous for implantation of the nucleus replacement device because, in the approximately linear state, the device's body may have a smaller cross-section than in its folded, non-linear state. A smaller cross-section may be desirable in order to facilitate insertion of the body into the confines of the intervertebral disc space. More particularly, insertion of the body into the intervertebral disc space through an incision or defect in the annulus may be facilitated by reducing the body's cross-section by unfolding it to its approximately linear state. A smaller cross-section also may be desirable in order to facilitate implantation of the foldable nucleus replacement device by the use of minimally invasive and minimal access surgical techniques.

In a preferred embodiment, the device's body is capable of folding upon itself more than two times. In a more preferred embodiment, the device's body is capable of folding upon itself seven times. It will be appreciated that, generally, for a given final volume of the device's body, the more times the device's body is capable of folding upon itself, the smaller the cross-section of the body when unfolded to an approximately linear state. The foldable nucleus replacement device therefore may provide improved handling and manipulation characteristics in that it may be deformed, straightened and otherwise handled by an individual without resulting in any significant breakage or other damage to the foldable nucleus replacement device.

The foldable nucleus replacement device's body preferably has shape memory. In a preferred embodiment, the fully folded, non-linear state of the device's body is its natural, resting, or relaxed state. Also, the unfolded, approximately linear state of the device's body is an activated state. Therefore, the nucleus replacement device preferably automatically will fold from its unfolded, approximately linear state to its folded, non-linear state unless restrained from doing so. Alternatively, as is known with shape memory materials, the device will fold upon application of energy, such as heat. For example, heat from the body into which the device is implanted may cause the device to fold. In this way, the foldable nucleus replacement device may be unfolded to facilitate implantation, and preferably automatically folds to its non-linear, relaxed state upon or during implantation. In other words, the foldable nucleus replacement device's body preferably is biased towards its folded, non-linear state.

Each fold in the body preferably defines an inner surface and an outer surface, relative to the fold. In order to promote deformation of the body, indents, grooves, or cutouts may be provided on the inner and outer surfaces of each fold.

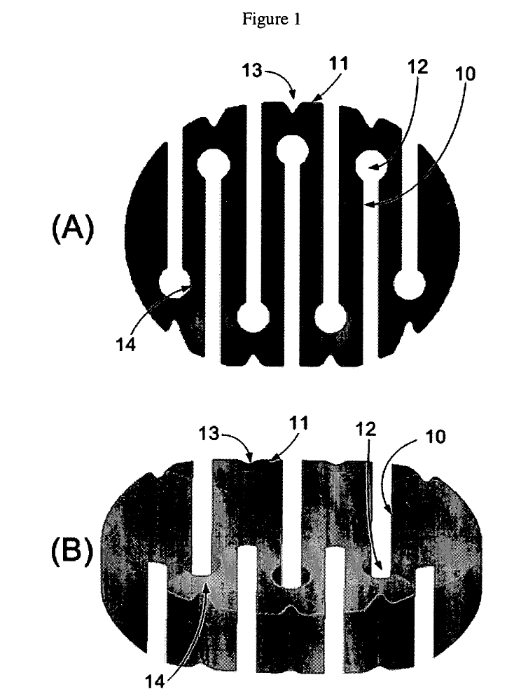

FIG. 1, embodiments A and B, illustrates an exemplary foldable nucleus replacement device. The device's body comprises numerous folds 14. Each fold defines an inner surface 10 and an outer surface 11. An indent or cutout 12 may be positioned at the inner surface of the folds. Also, an indent or groove 13 may be positioned at the outer surface of the folds. These indents, grooves, and cutouts advantageously may facilitate unfolding of the foldable nucleus replacement device's body for at least a temporary period of time without significant permanent deformation, cracking, tearing, breakage, or other damage to the device's body.

In certain preferred forms of the invention, the indents 12 and 13 positioned at the inner and outer surfaces, respectively, defined by the folds in the body of the foldable nucleus replacement device described herein have a radius of at least about 1 millimeter in the case of a cutout or indent 12, or a depth of about I millimeter in the case of an indent or grove 13. Moreover, in other preferred forms of the invention, a reinforcing material may be included at the inner and outer fold surfaces 10 and 11 to further improve the structural integrity of the foldable nucleus replacement device's body. The reinforcing material may be, for example, a fabric that is either woven, or non-woven, and may be formed from braided fibers for further strength. The reinforcing material may be positioned on the inner and outer fold surfaces, may project therefrom, or may be entirely embedded under the inner and outer surfaces defined by the folds.

In other embodiments, folding of the device's body may be facilitated by altering the composition of the materials comprising the body so that it is more flexible in certain pre-determined locations or positions than in the remainder of the body. In this way, the body may tend to fold at the more flexible pre-determined locations or positions upon insertion into the intervertebral disc space. Therefore, the pre-determined locations or positions of greater flexibility may be pre-determined folds. Another embodiment includes providing the increased flexibility by making the material thinner in the pre-determined locations. In another alternative embodiment, folding of the device at pre-determined locations or positions may be affected by reinforcing the body except at the pre-determined locations or positions. Reinforcing the body, as described herein, may make the reinforced portions or sections of the body less flexible. In this way, the body may tend to fold at the non-reinforced locations or positions. Therefore, the non-reinforced locations or positions may be pre-determined folds.

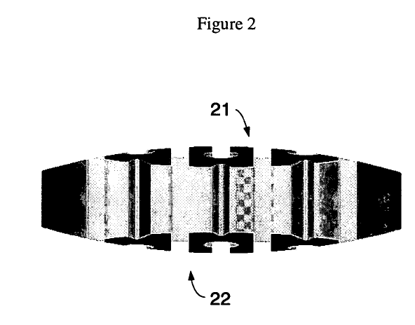

FIG. 2 illustrates an exemplary configuration of the top and bottom surfaces of the foldable nucleus replacement device. When folded, the device's body preferably defines top 21 and bottom 22 surfaces. The top and bottom surfaces of the device preferably each may be chosen in accordance with the geometry of the lower and upper vertebral endplates, respectively, that the top and bottom surfaces of the device may be in contact with when implanted in the intervertebral disc space. Therefore, if the vertebral endplate surfaces are flat, preferably the top and bottom surfaces of the foldable intervertebral disc device may be flat in order to properly engage the flat vertebral endplates. Alternatively, if the vertebral endplate surfaces are concave, the top and bottom surfaces of the foldable intervertebral disc device may be convex in order to properly engage the concave vertebral endplates. In FIG. 2, the exemplary top 21 and bottom 22 surfaces of the foldable nucleus replacement device are convex, which is a typical geometry for engaging vertebral body endplates.

As illustrated in FIG. 1, embodiment A, the foldable nucleus replacement device's body preferably assumes an oval-like shape when it is in a fully folded state. The position or location of the pre-determined folds in the device's body may be chosen in order to ensure an oval-like shape when the body is fully folded. Alternatively, by positioning or locating the pre-determined folds in the device's body at other positions or locations in the body, different shapes may be attained. For example, the body may be designed to fold into square or rectangular shapes instead of oval-like shapes.

In a preferred embodiment, the foldable nucleus replacement device's body folds into an oval-like shape. Preferably, the dimension of the major axis of the oval-like shape is from about 7 centimeters to about 13 centimeters. More preferably, the dimension of the major axis is from about 9 centimeters to about 11 centimeters. Most preferably, the dimension of the major axis is about 10 centimeters. Furthermore, the dimension of the minor axis of the oval-like shape preferably is from about 5 centimeters to about 11 centimeters. More preferably, the dimension of the minor axis is from about 7 centimeters to about 9 centimeters. Most preferably, the dimension of the minor axis is about 8 centimeters. Additionally, the height of the foldable nucleus replacement device preferably is from about 1 centimeter to about 5 centimeters at its apex. More preferably, the height is from about 2 centimeters to about 4 centimeters at its apex. Most preferably, the height is about 3 centimeters at its apex. Other dimensions may be chosen, for example, in order to obtain a proper fit of the foldable intervertebral disc device in the intervertebral disc space that is to be augmented or repaired.

The foldable nucleus replacement device may be used to fully or partially replace the natural, or native, nucleus pulposus in mammals, including humans and other animals. In one embodiment, the foldable nucleus replacement devices that are provided are configured to resist expulsion or other migration through a defect, or other opening, in the annulus fibrosis and to resist excessive migration within an intervertebral disc space. For example, in preferred forms of the invention the foldable nucleus replacement device's body is formed from a hydrogel or other hydrophilic or swellable material. While the foldable nucleus replacement device can pass through a relatively small incision in the annulus fibrosis when unfolded, the device also can expand following implantation and folding to substantially fill and conform to the intervertebral disc space.

In other embodiments, the foldable nucleus replacement device combines the advantages of an injectable/in-situ curing foldable nucleus replacement device with a pre-formed foldable nucleus replacement device. For example, a foldable nucleus replacement device may comprise a body surrounded by an outer, preferably resorbable or otherwise temporary, shell. The outer shell may advantageously anchor the body within the intervertebral disc space. The surface of the body may include various surface features, including various macro-surface patterns, and chemical or physical modifications to further enhance fixation of the body to the outer resorbable shell. The surface features, such as the macro-surface patterns and physical modifications, for example, also may enhance fixation of the foldable nucleus replacement device's body to surrounding tissues such that, in certain embodiments, no outer shell may be needed.

The outer surface of the outer shell preferably may conform to the shape of the intervertebral disc space and may completely surround the foldable nucleus replacement device's body. The outer shell may be useful to fill voids between the body of the foldable nucleus replacement device and the inner surfaces of the intervertebral disc space. This may aid in preventing expulsion from, or excessive migration in, the disc cavity of the body. Additionally, the inner surface of the outer shell preferably may conform to the shape of device, and preferably bonds to the outer surface of the body as discussed herein. In preferred embodiments, the body and outer shell of the foldable nucleus replacement device substantially fill the intervertebral disc space.

An outer shell may not only provide for a proper fit of the foldable nucleus replacement device within the intervertebral disc space for maximum load-bearing, stress transfer, and bonding of the foldable nucleus replacement device's surface to the surrounding disc tissues for fixation against excessive migration, it also may seal an annular defect for further resistance to migration and expulsion of the device. Such sealing of the annular defect also may provide additional physical and mechanical support to the intervertebral disc. Furthermore, the injectable outer shell material may provide intra-operative flexibility in fitting the body of the foldable nucleus replacement device within the intervertebral disc space as it may compensate for the differences in geometry and size between the disc space and the body. The optional outer shell also preferably is resorbable and, if so, preferably is replaced with tissue, such as fibrous tissue and including fibrous scar tissue, that may aid in permanently confining the load bearing foldable nucleus replacement device within the intervertebral disc space.

The dimensions of the foldable nucleus replacement device may vary depending on the particular case, and preferably are chosen for introduction into an intervertebral disc space. Moreover, the foldable nucleus replacement device preferably is wide enough to support adjacent vertebrae and is of a height sufficient to separate the adjacent vertebrae. In order to provide long-term mechanical support to the intervertebral disc, the volume of the foldable nucleus replacement device's body in the intervertebral disc space may be at least about 50%, preferably at least about 70%, further preferably at least about 80% and more preferably at least about 90% of the volume of the entire disc space. Preferably, the remaining volume may be occupied by the optional outer shell. The appropriate size of the foldable nucleus replacement device desired in a particular case may be determined by distracting the disc space to a desired level after the desired portion of the natural nucleus pulposus and any free disc fragments are removed, and measuring the volume of the distracted space, for example, with an injectable saline balloon.

The foldable nucleus replacement device's body may be formed from a wide variety of biocompatible polymeric materials, including elastic materials, such as elastomeric materials, hydrogels or other hydrophilic polymers, or composites thereof. Suitable elastomers include silicone, polyurethane, copolymers of silicone and polyurethane, polyolefins such as polyisobutylene and polyisoprene, neoprene, nitrile, vulcanized rubber, and combinations thereof. The vulcanized rubber described herein may be produced, for example, by a vulcanization process utilizing a copolymer produced as described, for example, in U.S. Pat. No. 5,245,098 from 1-hexene and 5-methyl-1,4-hexadiene.

Suitable hydrogels include natural hydrogels and those formed from polyvinyl alcohol; acrylamides such as polyacrylic acid and poly(acrylonitrile-acrylic acid); polyurethanes; polyethylene glycol; poly(N-vinyl-2-pyrrolidone); acrylates such as poly(2-hydroxy ethyl methacrylate) and copolymers of acrylates with N-vinyl pyrrolidone; N-vinyl lactams; acrylamide; polyurethanes; polyacrylonitrile; and so forth. The hydrogel materials further may be cross-linked to provide additional strength to the foldable nucleus replacement device's body. Examples of polyurethanes include thermoplastic polyurethanes, aliphatic polyurethanes, segmented polyurethanes, hydrophilic polyurethanes, polyether-urethane, polycarbonate-urethane, and silicone polyetherurethane. Other suitable hydrophilic polymers include naturally-occurring materials such as glucomannan gel, hyaluronic acid, polysaccharides such as cross-linked carboxyl-containing polysaccharides, and combinations thereof.

The nature of the materials employed to form the body may be selected so the formed bodies have sufficient load bearing capacity. In a preferred embodiment, a compressive strength of at least about 0.1 Mpa is desired, although compressive strengths in the range of from about 1 Mpa to about 20 Mpa are more preferred.

The optional outer shell may be formed from a wide variety of biocompatible, preferably elastic, elastomeric or deformable natural or synthetic materials, especially materials that are compatible with the foldable nucleus replacement device. The outer shell materials preferably remain in an uncured, deformable, or otherwise configurable state during positioning of the foldable nucleus replacement device in the intervertebral disc space, and should preferably rapidly cure, become harder or solidify after being introduced into the intervertebral disc space, or, in other embodiments, prior to positioning of the foldable nucleus replacement device in the intervertebral disc space. In preferred embodiments, the outer shell materials may remain deformable after they harden or otherwise solidify.

Suitable materials that may be used to form the outer shell include tissue sealants or adhesives made from natural or synthetic materials including, for example, fibrin, albumin, collagen, elastin, silk and other proteins, polyethylene oxide, cyanoacrylate, polylactic acid, polyglycolic acid, polypropylene fumarate, tyrosine-based polycarbonate, and combinations thereof. Other suitable materials include demineralized bone matrix. These precursor materials may be supplied in liquid, solution, or solid form, including gel form.

The foldable nucleus replacement device's body may include a variety of surface features on its outer surface, including chemical modifications and surface configurations, to provide surface features that advantageously improve the bonding between the outer surface of the body and the inner surface of the optional outer shell or, if an outer shell is not used, the inner surfaces of the intervertebral disc space. In one embodiment, the outer surface may be chemically modified utilizing, for example, chemical groups that are compatible with the materials used to form the optional outer shell. Suitable chemical modifications include, for example, surface grafting of reactive functional groups, including hydroxyl, amino, carboxyl and organofunctional silane groups. The groups may be grafted by methods known to the skilled artisan. Other modifications include pre-coating with a primer, preferably one that is compatible with the outer shell material, such as a layer of adhesive, sealing, or other materials used for forming the optional outer shell.

In yet another embodiment, the foldable nucleus replacement device's body may include a wide variety of surface configurations, such as macro-surface patterns, or protuberances. The macro-surface patterns preferably may be formed during formation of the body. However, the outer surface of the body also may be physically modified after formation of the foldable nucleus replacement device by, for example, laser drilling or thermal deformation. Physical modifications include, for example, a microtexturized surface formed by bead-blasting, plasma etching, or chemical etching. Procedures for modifying various surfaces in this manner are well known in the art.

In certain embodiments, the foldable nucleus replacement device's body may include only one or more of the outer surface features as described above, without the optional outer resorbable shell. The surface features may provide a certain level of fixation to the surrounding tissues of the intervertebral disc space for improved resistance to migration and/or expulsion.

In other embodiments, the foldable nucleus replacement device may comprise a supporting, or otherwise constraining, member wherein the supporting member is surrounded by an outer shell as described herein. For example, the supporting member may be a flexible, peripheral supporting band that is disposed circumferentially about the foldable nucleus replacement device's body, leaving the upper and lower surfaces of the body free from the supporting band. In another embodiment, the foldable nucleus replacement device may be reinforced with a supporting member that takes the form of a jacket. The jacket preferably completely surrounds the foldable nucleus replacement device's body.

Suitable supporting members, including reinforcing outer bands, covers, and other jackets, may be formed from a wide variety of biocompatible polymers, metallic materials, or a combination of materials that forms a strong but flexible support to prevent excessive deformation, including lateral (horizontal) deformation, of the foldable nucleus replacement device's body under compressive loading. Suitable materials for the supporting members include non-woven, woven, braided, and fabric materials made from polymeric fibers including, but not limited to, cellulose, polyethylene, polyester, polyvinyl alcohol, polyacrylonitrile, polyamide, polytetrafluorethylene, polyparaphenylene terephthalamide, and combinations thereof. Other suitable materials include non-reinforced or fiber-reinforced elastomers such as silicone, polyurethane, copolymers of silicone and polyurethane, polyolefins, including polyisobutylene and polyisoprene, neoprene, nitrile, vulcanized rubber, and combinations thereof. In a preferred embodiment, a combination, or blend, of silicone and polyurethane is used. Supporting members may be advantageously made from a porous material that, in the case of a body made from a hydrogel or other hydrophilic material, allows fluid circulation through the body to enhance pumping actions of the intervertebral disc. Supporting members further may be formed from carbon fiber yarns, ceramic fibers, metallic fibers or other similar fibers as described, for example, in U.S. Pat. No. 5,674,295.

The foldable nucleus replacement device's body may be covered by the jacket supporting member, or the band supporting member may be wrapped around the circumference of the body. In an embodiment where the body is formed from a hydrogel, or similar hydrophilic material, the hydrogel or other hydrophilic material preferably may be dehydrated a desired amount prior to being covered by the jacket, or prior to wrapping the band around the circumference of the body. The hydrogel body may be exposed to saline outside of the body, or may be inserted into the disc space and then exposed to body fluids in situ. Exposure to saline solution, body fluids, and water may cause the body to absorb water and swell. In reference to the peripheral band supporting member, the swelling or expansion of the hydrogel body in the horizontal direction is controlled by the amount of slack in the band. After the limited allowable horizontal expansion is reached, the body is forced to expand mostly in the vertical direction until reaching equilibrium swelling under the in vivo load. As the upper and lower surfaces of the body are not substantially constrained, the vertical expansion is mainly controlled by the applied stress and the behavior of the hydrogel material.

When the foldable nucleus replacement device's body is formed from an elastic material, such as a hydrogel, or other similar hydrophilic material, or when the foldable nucleus replacement includes the resorbable outer shell, it may advantageously deliver desired pharmacological agents. The pharmacological agent may be a growth factor that may advantageously repair the endplates and/or the annulus fibrosis. For example, the growth factor may include a bone morphogenetic protein, transforming growth factor-beta (TGF-beta), insulin-like growth factor, platelet-derived growth factor, fibroblast growth factor or other similar growth factor or combination thereof having the ability to repair the endplates and/or the annulus fibrosis of an intervertebral disc.

The growth factors are typically included in the foldable nucleus replacement devices in therapeutically effective amounts. For example, the growth factors may be included in the foldable nucleus replacement devices in amounts effective in repairing an intervertebral disc, including repairing the endplates and the annulus fibrosis. Such amounts will depend on the specific case, and may thus be determined by the skilled artisan, but such amounts may typically include less than about 1% by weight of the growth factor. The growth factors may be purchased commercially or may be produced by methods known to the art. For example, the growth factors may be produced by recombinant DNA technology, and may preferably be derived from humans. As an example, recombinant human bone morphogenetic proteins (rhBMPs), including rhBMP 2-14, and especially rhBMP-2, rhBMP-7, rhBMP-12, rhBMP-13, and heterodimers thereof may be used. However, any bone morphogenetic protein is contemplated including bone morphogenetic proteins designated as BMP-1 through BMP-18.

BMPs are available from Genetics Institute, Inc., Cambridge, Mass. and may also be prepared by one skilled in the art as described in U.S. Pat. No. 5,187,076 to Wozney et al.; U.S. Pat. No. 5,366,875 to Wozney et al.; U.S. Pat. No. 4,877,864 to Wang et al.; U.S. Pat. No. 5,108,922 to Wang et al.; U.S. Pat. No. 5,116,738 to Wang et al.; U.S. Pat. No. 5,013,649 to Wang et al.; U.S. Pat. No. 5,106,748 to Wozney et al.; and PCT Patent Nos. WO 93/00432 to Wozney et al.; WO 94/26893 to Celeste et al.; and WO 94/26892 to Celeste et al. All bone morphogenic proteins are contemplated whether obtained as above or isolated from bone. Methods for isolating bone morphogenetic protein from bone are described, for example, in U.S. Pat. No. 4,294,753 to Urist and Urist et al., 81 PNAS 371, 1984. The disclosures of each of the aforementioned U.S. patents are incorporated by reference in their entireties.

In other forms of the invention, the pharmacological agent may be one used for treating various spinal conditions, including degenerative disc disease, spinal arthritis, spinal infection, spinal tumor and osteoporosis. Such agents include antibiotics, analgesics, anti-inflammatory drugs, including steroids, and combinations thereof. Other such agents are well known to the skilled artisan. These agents are also used in therapeutically effective amounts. Such amounts may be determined by the skilled artisan depending on the specific case.

The pharmacological agents preferably are dispersed within the hydrogel, or other hydrophilic, material of the foldable nucleus replacement device's body for in vivo release, and/or, with respect to the foldable nucleus replacement devices with the resorbable outer shell, may be dispersed in the outer shell. The hydrogel can be cross-linked chemically, physically, or by a combination thereof, in order to achieve the appropriate level of porosity to release the pharmacological agents at a desired rate. The agents may be released upon cyclic loading, and, in the case of foldable nucleus replacement devices including a resorbable outer shell, upon resorption of the shell. The pharmacological agents may be dispersed in the foldable nucleus replacement devices by adding the agents to the solution used to form the foldable nucleus replacement device's body, by soaking the formed body in an appropriate solution containing the agent, or by other appropriate methods known to the skilled artisan. Alternatively, the pharmacological agents may be coated on the surface of the device's body or the surface of the optional outer shell. For example, the agents may be chemically attached to the outer surface of the body or the outer surface of the optional outer shell.

The foldable nucleus replacement devices described herein also may be marked for x-ray identification by having small metal beads or wire embedded therein.

This may aid in positioning and orientation of the nucleus replacement device within the intervertebral disc space during implantation. For example, a surgeon or other professional may use fluoroscopic imaging to identify the marked foldable nucleus replacement devices in the intervertebral disc and then re-position or re-orient the device accordingly, if needed.

Methods of forming the foldable nucleus replacement devices described herein also are provided. In one embodiment, with respect to a foldable nucleus replacement device having an optional outer shell, the foldable nucleus replacement device may be formed by first forming the body and then forming the outer shell. Methods of forming the body are well known in the art.

For example, if the body is made of elastomeric materials, such as powdered elastomers including, for example, styrene/ethylene/butylene block copolymers, the powdered elastomer may be placed into an appropriate mold and may be compressed and heated to melt the powder. The mold then may be cooled to room temperature. If the foldable nucleus replacement device is made from a hydrogel, such as a polyvinyl alcohol, the polyvinyl alcohol powder may be mixed with a solvent, such as, for example, water or dimethylsulfoxide, or combinations thereof, and heated and shaken until a uniform solution is formed. The solution then may be poured into a mold, such as a rubber mold, and may be cooled at an appropriate temperature, such as about 0° Celsius to about −80° Celsius, for several hours to allow for crystallization. After cooling, the hydrogel can be partially or completely hydrated by soaking and rinsing with water but, in certain preferred embodiments, may remain dehydrated so that it may be inserted into the intervertebral disc space through a smaller aperture in the annulus fibrosis.

In another embodiment, a method for implanting a foldable nucleus replacement device is provided. In one embodiment, a foldable nucleus replacement device as described herein is provided. The foldable nucleus replacement device may be deformed by, for example, manual force into an approximately linear, non-relaxed state for insertion through an aperture in the annular fibrosis. The aperture may be a defect formed through deterioration or other injury to the annulus fibrosis, or may be made by purposely incising the annulus. Deforming the body to its approximately linear state may be desirable in order to reduce the body's cross-section to facilitate insertion. Reduction of the body's cross-section may be especially desirable if minimally invasive or minimal access surgical techniques are to be used during delivery and implantation of the device.

A portion, or substantially all, of the natural nucleus pulposus may be removed from the intervertebral disc space, depending on the circumstances, prior to introduction of the foldable nucleus replacement device into the intervertebral disc space. An incision may be made in the annulus fibrosis, or one may take advantage of a defect in the annulus, in order to remove the natural nucleus pulposus and any free disc fragments within the intervertebral disc space.

Additionally, it may be desirable to measure the volume and other dimensions of the intervertebral disc space before inserting the device. This may allow a device to be chosen having an appropriate size and geometry for the intervertebral disc that is to be augmented by implantation of the nucleus replacement device.

The foldable nucleus replacement device then may be positioned in a delivery tool, such as that described in U.S. Pat. No. 5,716,416, and inserted through the aperture in the annulus. The disclosure of U.S. Pat. No. 5,716,416 is incorporated by reference herein in its entirety. As the foldable nucleus replacement device enters the intervertebral space and is no longer subject to manual force, it may deform back into its folded, non-linear state. If the outer shell precursor material was already placed in the intervertebral disc space, excess precursor material may flow out of the disc space. This excess material may be promptly removed before it sets or otherwise cures. The outer shell material may be injected, or otherwise introduced, into the disc space utilizing devices that are well known in the art, such as syringes, sealant/caulk guns, automatic liquid injectors, and applicators that include, for example, two separate syringes which allow for simultaneous mixing of the components in a static mixer and delivery to the site, and may be injected either prior to or after introduction of the foldable nucleus replacement device's body into the disc space.

Whether the outer shell material is introduced prior to or after introduction of the body into the disc space, the distractor then may be removed, any excess precursor material seeping out of the disc space also may be removed and the precursor material within the disc space may be cured to form the outer shell. It is noted that, alternatively, the foldable nucleus replacement device's body already may be surrounded by the outer shell, which may be in a partially or fully hardened state but preferably remains deformable, prior to introducing the foldable nucleus replacement device into the intervertebral disc space.

The foregoing detailed description is provided to describe the invention in detail, and is not intended to limit the invention. Those skilled in the art will appreciate that various modifications may be made to the invention without departing significantly from the spirit and scope thereof.

What is claimed is:

1. A foldable nucleus replacement device comprising a body capable of folding upon itself more than twice at pre-determined folds, each fold defining an inner surface and an outer surface; wherein the body also is capable of unfolding to an approximately linear state before implantation, and returning to its folded, non-linear state after implantation.

2. The device of claim 1, wherein the folded, non-linear state corresponds to a relaxed state, and the approximately linear state corresponds to a less relaxed state.

3. The device of claim 1, wherein the body is capable of folding on itself seven times.

4. The device of claim 1, further comprising an indent or cutout positioned on the inner surface of each fold.

5. The device of claim 1, further comprising an indent or groove positioned on the outer surface of each fold.

6. The device of claim 1, wherein the nucleus replacement device assumes an oval-like shape when it is in a fully folded, non-linear state.

7. The device of claim 1, wherein the body, in its folded, non-linear state, defines a top surface and a bottom surface; and wherein the top surface and bottom surface each individually has a shape that is selected from flat and convex.

8. The device of claim 1, further comprising at least one reinforcing member secured to or embedded in the body.

9. The device of claim 8, wherein the reinforcing member is selected from the group consisting of a supporting jacket, a supporting band, and fibers embedded in the body.

10. The device of claim 1, further comprising at least one pharmacological agent dispersed within the body.

11. The device of claim 1, further comprising at least one pharmacological agent coated on the surface of the body.

12. The device of claim 1, further comprising a resorbable shell encasing the body.

13. The device of claim 12, wherein the resorbable shell is formed in vivo.

14. The device of claim 12, wherein the resorbable shell comprises a material selected from the group consisting of fibrin, albumin, gelatin, collagen, elastin, silk, demineralized bone matrix, polyethylene oxide, polyethylene glycol, polyvinyl alcohol, polypropylene fumarate, and mixtures and combinations thereof.

15. The device of claim 12, further comprising at least one pharmacological agent dispersed within the resorbable shell.

16. The device of claim 1, further comprising at least one surface modification of the body.

17. The device of claim 16, wherein the surface modification is selected from the group consisting of microtexturization and the attachment of reactive chemical groups to the body.

18. The device of claim 1, wherein the body comprises a material selected from the group consisting of hydrogels, elastomers, thermosetting polymers, thermoplastic polymers, and thermoplastic elastomers.

19. A method for replacing at least a portion of an intervertebral disc nucleus, comprising:
providing a foldable nucleus replacement device as in claim 1;
unfolding the foldable nucleus replacement device to an approximately linear state;
inserting the unfolded foldable nucleus replacement device into an intervertebral disc space; and
allowing the nucleus replacement device to re-fold during insertion.

20. The method of claim 19, further comprising contacting the foldable nucleus replacement device with a resorbable polymer following insertion into the intervertebral disc space.

21. The method of claim 19, further comprising surgically evacuating at least a portion of the nucleus pulposus material and any free disc fragments from the intervertebral disc space prior to inserting the unfolded foldable nucleus replacement device.

22. The method of claim 19, further comprising measuring the volume of the intervertebral disc space prior to inserting the unfolded nucleus replacement device.

23. The method of claim 19, wherein inserting the unfolded nucleus replacement device is carried out by minimally invasive surgical techniques.

24. The method of claim 19, wherein inserting the unfolded nucleus replacement device is carried out by minimal access surgical techniques.

25. The method of claim 19, wherein the unfolded nucleus replacement device is inserted through an incision or a defect in the annulus.
